(19) United States
(12) Patent Application Publication
(10) Pub. No.: US 2002/0143244 A1
(43) Pub. Date: Oct. 3, 2002

(54) In-vivo blood storage system

(75) Inventors: Kenneth Kensey, Malvern, PA (US); William N. Hogenauer, Gilbertsville, PA (US)

Correspondence Address: CAESAR, RIVISE, BERNSTEIN,, COHEN & POKOTILOW, LTD., 12TH FLOOR, SEVEN PENN CENTER, 1635 MARKET STREET, PHILADELPHIA, PA, 19103-2212, US

(73) Assignee: Rheologics, Inc., Exton, PA

(21) Appl. No.: 10/068,345

(22) Filed: Feb. 5, 2002

Related U.S. Application Data
(60) Provisional application No. 60/279,601, filed on Mar. 29, 2001.

(51) Int. Cl. A61B 5/00; G01N 11/00

(52) U.S. Cl. 600/370; 73/54.01; 422/50

(57) ABSTRACT

A method and apparatus for testing the efficacy of agents (e.g., new drugs, old drugs, biologically-active agents, etc.) by removing a predetermined amount of blood from a living being and placing this predetermined amount of blood in continuous motion in a temperature-controlled environment and then removing samples from the continuously-moving predetermined amount of blood. The drug, or drugs, or agents under test can then be administered to the removed samples, instead of into the living being, thereby avoiding subjecting the living being to any risk of any adverse reaction while utilizing blood samples that simulate the circulating blood of the living being.

### FIELD OF THE INVENTION

The invention pertains to methods and apparatus for testing the efficacy of agents on blood, and more particularly, to methods and apparatus for simulating blood flow using a sample of blood, outside of the body, while administering agents thereto and monitoring the effects of such agents on the blood sample.

### BACKGROUND OF INVENTION

The testing of agents (e.g., drugs, biologically-active agents, etc.) to determine the their effects (e.g., toxic levels, therapeutic levels, etc.) on the blood of a living being (e.g., reducing the viscosity of the blood) usually involves administering small amounts of the agent to a living being, e.g., a human patient, and then observing the patient's vital signs and any other biological or molecular reaction. It is important, especially with regard to determining the effects of the agent on a living being's blood viscosity, to have the agent administered into the circulating blood of the living being rather than having the agent administered into a static sample of blood removed from the patient and then observing any changes in the blood sample.

However, such a procedure not only may subject a patient to risk of a severe reaction, but it is tedious in preparing the patient and is inconvenient to the patient who must remain under observation and/or restricted behavior until the study is complete.

Thus, there remains a need for an apparatus and method for testing the efficacy of an agent on the circulating blood of a living being while avoiding the risk of the living being suffering a severe reaction to the drug, as well as the inconvenience to the living being.

### SUMMARY OF THE INVENTION

A method for testing the efficacy of an agent (e.g., a drug, biologically-active agents, etc.) on the circulating blood of a living being (e.g., a human being). The method comprises the steps of: (a) removing a predetermined amount of blood (e.g., 50-100 cc) from the living being into a collector (e.g., an intravenous bag that is uncoated or coated with a biocompatible coating); (b) placing the collector containing the predetermined amount of blood in continuous motion such that the predetermined amount of blood is continuously moving within the collector; (c) maintaining the temperature of the collector containing the predetermined amount of blood at the temperature of the body of the living being while such blood is in the continuous motion; and (d) removing a portion (e.g, 10 cc) of the predetermined amount of blood for administering the agent thereto and immediately analyzing the effect of the agent on the portion of the predetermined amount of blood.

An apparatus for storing blood of a living being (e.g., a human being) outside of the body of the living being while simulating the movement and temperature of the circulating blood. The apparatus comprises: a collector (e.g., an intravenous bag) adapted for receiving a predetermined amount of blood (50-100 cc) from the living being and whereby the collector comprises an internal biocompatible coating (e.g., phosphoryl choline); a motion device (e.g, a swivel rocker, a reciprocating arcuate motion generator, a reciprocating linear motion generator, etc.) upon, or in, which is positioned the collector and whereby the motion device provides continuous motion to the collector; a temperature-controlled unit (e.g., an incubator) in which is positioned the collector and motion device and whereby the temperature-controlled unit maintains the collector at the body temperature of the living being.

### DESCRIPTION OF THE DRAWINGS

FIG. 1 is an isometric view, in partial view, of the in vivo blood storage system;

FIG. 2 is a side elevational view, partially in section, of the present invention using an alternative motion device;

FIG. 3 is a side elevational view, partially in section, of the present invention using another motion device;

FIG. 4 is a functional diagram of a dual riser/single capillary tube viscometer in accordance with application Ser. No. 09/573,267; and

FIG. 5 is a functional diagram of a mass detection capillary viscometer in accordance with application Ser. No. 09/789,350.

### DETAILED DESCRIPTION OF THE PREFERRED EMBODIMENTS

Referring now in detail to the various figures of the drawing wherein like reference characters refer to like parts, there is shown at 120 in FIG. 1, an in-vivo blood storage system. The system 120 basically comprises a motion device 122, a collector 124 (e.g., an intravenous, IV, bag) disposed on or in the motion device 122 and, both of which, are positioned in a temperature-controlled unit 126 (e.g., an incubator). Although not shown, the interior of the collector 124 comprises a biocompatible coating (e.g., an anti-thrombogenic material, such as Heparin, and/or anti-thrombolytic coatings, e.g. phosphoryl choline, etc., available from Biocompatibles, Ltd., Uxbridge, UK) to replicate the interior of the blood vessel tissue. The motion device 122 operates continuously to effect the high and low shear rates that are experienced by the circulating blood as it travels through the living being's blood vessels. The temperature-controlled unit 126 operates to maintain the temperature of the blood in the collector 124 at the temperature of the living being's body.

In use, the empty collector 124 is positioned on or in the motion device 122 which is energized to provide the continuous movement; in addition, the temperature-controlled unit 126 is also energized to generate the body temperature of the living being. With the motion device 122 and temperature-controlled unit 126 operating and at the proper temperature, a predetermined amount of blood 121 (e.g., 50-100 cc) is diverted into the collector 124 via a lumen (e.g., a catheter/needle inserted into blood vessel of the living being), not shown; the lumen is coupled to the collector 124 via a self-sealing surface 125 (e.g., those used in serum containers into which a needle is pushed). Once the collector 124 is filled with the predetermined amount of blood 121, the lumen is disengaged from the living being and the collector 124. At this point, there is now a continuously-moving portion of the living being's blood that is available for testing.

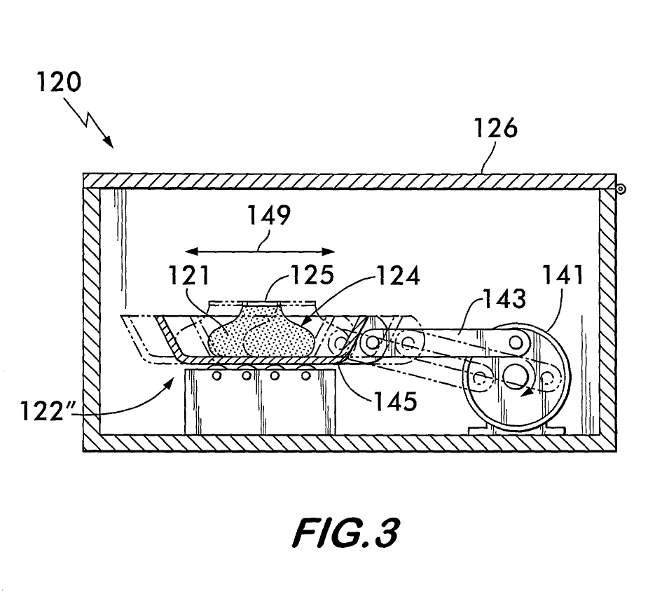

An example of such testing may comprise the following. It may be desirable to determine the efficacy of a certain new drug, or agent, on changing the viscosity of the living being's blood. To that end, the clinician removes a sample of blood (e.g., 10 cc), not shown, from the collector 124 via the self-sealing surface 125 and then administers the new drug or agent. This mixture is then immediately deposited into a blood viscometer that moves the mixture through a plurality of shear rates; such viscometers are disclosed in application Ser. No. 091573,267entitled DUAL RISER/SINGLE CAPILLARY and application Ser. No. 09/789,350 entitled MASS DETECTION CAPILLARY VISCOMETER, both of which are assigned to the same Assignee as the present invention, namely Rheologics, Inc. (formerly Visco Technologies, Inc.), and both of whose entire disclosures are incorporated by reference herein. Other suitable viscosity measuring apparatus may be used in lieu of these apparatus. In particular, FIG. 3 depicts a viscometer 20 disclosed in application Ser. No. 09/573,267. The mixture is deposited into the upper end 128 of one of a riser tube R1 of the viscometer 20. In accordance with the operation of the viscometer 20, a failing column 82 of the mixture is generated in the riser tube R1 while a rising column of the mixture 84 is generated in a second riser tube R2. This movement is analyzed by the viscometer 20 in accordance with application Ser. No. 09/573,267 and the viscosity of the mixture determined. This is compared to the viscosity of the blood before the drug or agent is administered.

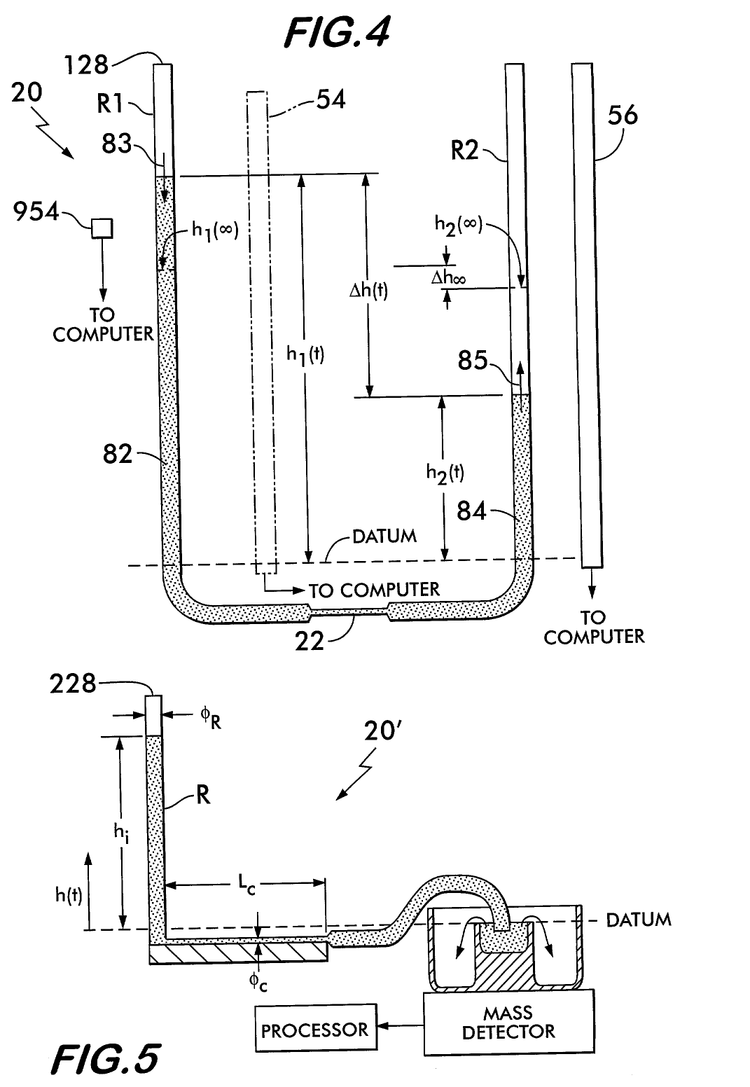

Alternatively, as shown in FIG. 4, the mixture can be deposited into the open end 228 of the riser tube R of the viscometer 20′. In accordance with the operation of the viscometer 20′, a falling column of the mixture is generated in the riser tube R and the increasing mass of the collected mixture detected by the mass detector. This increasing mass is analyzed by the viscometer 20′ in accordance with application Ser. No. 09/789,350 and the viscosity of the mixture determined. This is compared to the viscosity of the blood before the drug or agent is administered.

As shown in FIG. 1, the motion device 122 may comprise a rocker device using three-dimensional motion. For example, the motion device 122 may comprise the Cole Parmer® Rocker with Three-Dimensional Motion, part number EW-51702-05. In accordance with such operation, each corner of the support plate 127 is serially moved into a downward position, thereby causing the three-dimensional motion.

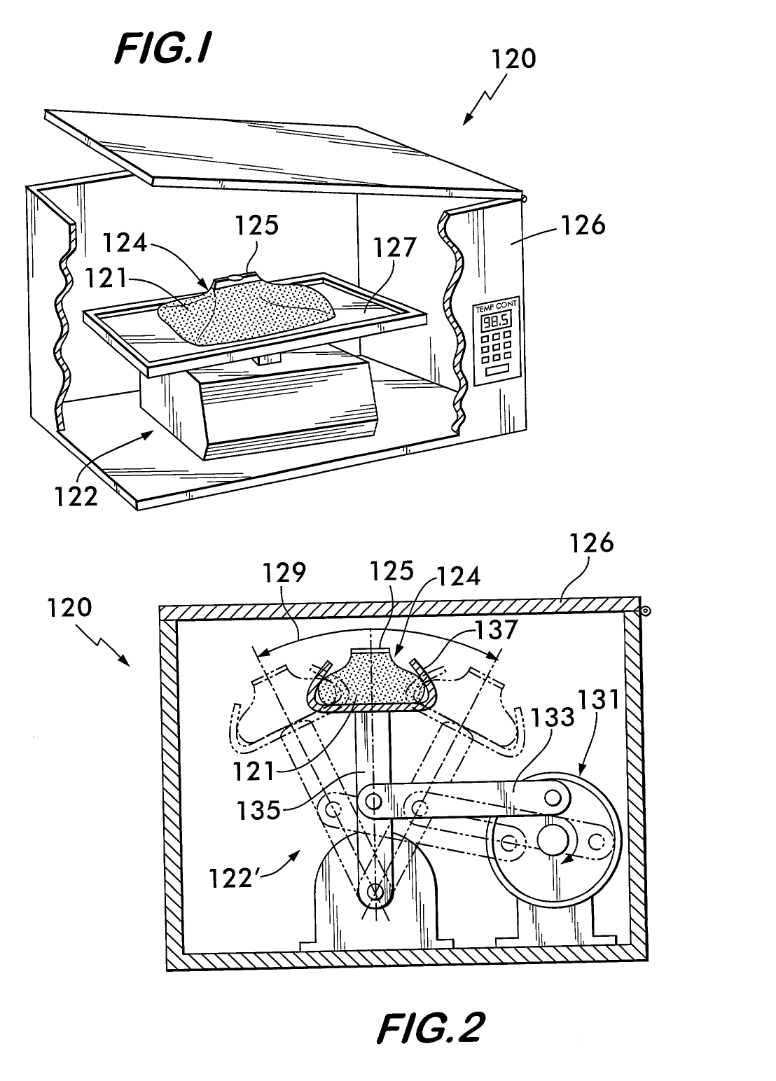

Alternatively, as shown in FIG. 2, the motion device 122 may comprise the device 122′ that generates a reciprocating arcuate motion 129. For example, a motor 131 drives one end of a clevis 133 having another end attached to an intermediate point on a support member 135. At the top of the support member 135 is a collector support surface 137. As the motor 131 rotates, the collector 124 is subjected to the reciprocating arcuate motion 129.

Furthermore, as shown in FIG. 3, the motion device 122 may comprise a linear motion device 122″ that generates reciprocating linear motion 149. For example, a motor 141 drives one end of a clevis 143 having another end attached to one end of a collector support surface 145. As the motor 141 rotates, the collector 124 is subjected to the reciprocating linear motion 149.

The incubator 126 may comprise the Cole Parmer® EW-39401-05 High-Performance Incubator. The enclosure of the incubator 126 may also provide a darkened environment which is similar to the blood vessels through which the circulating blood normally flows.

It should be understood that although one agent or drug is mentioned as being administered to the sample removed from the collector 124, it is within the broadest scope of the present invention to include a plurality of agents or drugs being administered to the sample.

It should also be understood that the analysis that the sample removed from the collector 124 is subjected to is not limited to viscosity determination. Rather, the sample can be analyzed for an entire battery of tests or screening. Whatever test/screening is conducted, that test/screening is immediately conducted once the sample is removed from the collector 124.

It should be noted that, if desired, an adulterant may be added to the interior of the collector to ensure that the blood remains fluid and does not clot to any extent which would prevent a viable measurement of its viscosity.

Moreover, the subject invention isn't limited to the particular mechanisms for keeping the blood in motion, as discussed above. Thus any suitable means for keeping the blood from stasis are contemplated by this invention, e.g., pulsatile pumps, rotary pumps, mechanical mixers, agitators, etc.

Without further elaboration, the foregoing will so fully illustrate our invention that others may, by applying current or future knowledge, readily adopt the same for use under various conditions of service.

We claim:

1. A method for testing the efficacy of an agent on the circulating blood of a living being, said method comprising the steps of:
(a) removing a predetermined amount of blood from the living being into a collector that is coated with a biocompatible coating;
(b) placing said collector containing said predetermined amount of blood in continuous motion such that said predetermined amount of blood is continuously moving within said collector;
(c) maintaining the temperature of said collector containing said predetermined amount of blood at the temperature of the body of the living being while such blood is in said continuous motion; and
(d) removing a portion of said predetermined amount of blood for administering the agent thereto and immediately analyzing the effect of the agent on said portion of said predetermined amount of blood.

2. The method of claim 1 wherein said step of placing said collector containing said predetermined amount of blood in continuous motion comprises positioning said collector containing said predetermined amount of blood on a motion device that maintains the predetermined amount of blood in continuous motion within the collector.

3. The method of claim 2 wherein said motion device comprises a swivel rocker that subjects said collector containing said predetermined amount of blood to three-dimensional motion.

4. The method of claim 2 wherein said motion device comprises an arm that moves said collector containing said predetermined amount of blood in a reciprocating arcuate motion.

5. The method of claim 2 wherein said motion device comprises a surface that moves said collector containing said predetermined amount of blood in a reciprocating linear motion.

6. The method of claim 1 wherein said step of immediately analyzing the effect of the agent on said portion of said predetermined amount of blood comprises disposing said portion into a viscometer that moves said portion throughout a plurality of shear rates for determining the viscosity of said portion of said predetermined amount of blood.

7. The method of claim 6 wherein said viscometer comprises a U-shaped structure wherein said portion that is disposed therein forms a falling column of fluid and a rising column of fluid and wherein the movement of at least one of said columns is monitored over time.

8. The method claim 6 wherein said viscometer comprises an L-shaped structure wherein said portion that is disposed therein forms a falling column of fluid and wherein the fluid flows into a collector whose changing mass is monitored over time.

9. An apparatus for storing blood of a living being outside of the body of the living being while simulating the movement and temperature of the circulating blood, said apparatus comprising:
a collector adapted for receiving a predetermined amount of blood from the living being, said collector comprising an internal biocompatible coating;
a motion device upon, or in, which is positioned said collector, said motion device providing continuous motion to said collector;
a temperature-controlled unit in which is positioned said collector and motion device, said temperature-controlled unit maintaining said collector at the body temperature of the living being.

10. The apparatus of claim 9 wherein said collector comprises an intravenous bag.

11. The apparatus of claim 9 wherein said motion device comprises a swivel rocker that subjects said collector to three-dimensional motion.

12. The apparatus of claim 9 wherein said motion device comprises:
a motor;
a clevis having one end coupled to said motor;
a collector support member having a lower end that is pivotal about a horizontal axis and having a collector support surface at an upper end for holding said collector, said clevis having a second end coupled to an intermediate point between said upper and lower ends on said collector support member; and
said motor rotating continuously to cause said collector support surface to move in reciprocating arcuate motion.

13. The apparatus of claim 9 wherein said motion device comprises:
a motor;
a clevis having one end coupled to said motor;
a collector support surface for holding said collector, said clevis having a second end coupled to a side of said collector support surface; and
said motor rotating continuously to cause said collector support surface to move in reciprocating linear motion.

14. The apparatus of claim 9 wherein said temperature-controlled unit is an incubator.

15. The apparatus of claim 9 wherein said collector comprises a self-sealing material that permits a sample of blood to be removed from said collector for mixing with at least one agent while maintaining a sealed closure of said predetermined amount of blood.

16. The apparatus of claim 9 wherein said biocompatible coating comprises phosphoryl choline.
